(19) United States
(12) Patent Application Publication
(10) Pub. No.: US 2002/0115965 A1
(43) Pub. Date: Aug. 22, 2002

(54) Safety intravenous set

(76) Inventors: Yang-soo Jang, Kwangju-city (KR); Mi-kyeong Jin, Kwangju-city (KR); Mun-seok Jang, Kwangju-city (KR)

Correspondence Address: BURNS DOANE SWECKER & MATHIS L L P, POST OFFICE BOX 1404, ALEXANDRIA, VA, 22313-1404, US

(21) Appl. No.: 10/066,341

(22) Filed: Feb. 5, 2002

(30) Foreign Application Priority Data
Feb. 9, 2001 (KR) U2001/0003113

(51) Int. Cl. A61M 5/14; A61M 5/00

(52) U.S. Cl. 604/254

(57) ABSTRACT

Disclosed is a safety intravenous set in which a spherical ball is provided in a transparent barrel for confirming a flow of the Ringer's solution to easily float up due to its own buoyancy when the Ringer's solution is injected so that the Ringer's solution is facilely administered through a supplying tube, and also, when the administration of the Ringer's solution is completed, the spherical ball automatically blocks an outlet port so as to efficiently prevent a back flow of blood of the patient. The transparent barrier has a double-curbed structure at a lower portion thereof, and also has a hemispherical settling portion for easily floating up the spherical ball when the Ringer's solution is introduced into the transparent barrier.

### BACKGROUND OF THE INVENTION

1. Field of the Invention

The present invention relates to an intravenous set for administering Ringer's solution in a blood vessel of a patient, and more particularly, to a safety intravenous set in which a spherical ball is provided in a transparent barrel for confirming a flow of the Ringer's solution to easily float up due to its own buoyancy when the Ringer's solution is injected so that the Ringer's solution is facilely administered through a supplying tube, and also, when the administration of the Ringer's solution is completed, the spherical ball automatically blocks an outlet port so as to efficiently prevent a back flow of blood of the patient.

2. Description of the Related Art

Generally, in case digestive power of a patient is lowered or there is a need to supply a nutritious substance, i.e., glucose or an injection to the patient, Ringer's solution contained in a Ringer pack or bottle is administered through a blood vessel to the patient.

FIG. 6 is a cross-sectional view of a conventional safety intravenous set. As shown in FIG. 6, a transparent barrel 100 is gradually narrowed from an upper portion toward a lower portion thereof, and has a curved portion 110 and a spherical ball 200 which floats up due to its own buoyancy when the Ringer's solution is injected therein and blocks an outlet port 120 when the Ringer's solution is completely injected.

In the conventional intravenous set, as described above, the Ringer's solution is supplied through a needle formed at an end of a supplying tube 300 in a status that a needle rod 400 of the transparent barrel 100 is inserted into a rubber cork of a Ringer bottle 500. At this time, the Ringer's solution is introduced through an inlet port 410 to the transparent barrel 100. The spherical ball 200 provided in the transparent barrel 100 is floated up by its own buoyancy. Therefore, the Ringer's solution passes through the outlet port 120 and the supplying tube 300 and is administered into the body of the patient.

If the Ringer's solution in the Ringer pack or bottle is completely injected in the body, the spherical ball 200 in the transparent barrel 100 is slowly settled down at the lower portion so as to block the outlet port 120 and thus close the supplying tube 300.

However, in the conventional safety intravenous set, as described above, when the Ringer's solution is introduced into the transparent barrel 100 in an early state, since the spherical ball 200 is settled at the lower portion of the transparent barrel to be closely contacted with an inner side of the transparent barrel, even though the Ringer's solution is introduced into the transparent barrel, the spherical ball 200 may not easily float up due to static frictional force between the spherical ball 200 and the inner side of the transparent barrel. Therefore, there is a problem that the Ringer's solution cannot be administered to the patient. Particularly, since medical implements for treating a person's life requires high reliability, such an erroneous operation of the medical implement is a very serious problem.

### SUMMARY OF THE INVENTION

Therefore, it is an object of the present invention to provide a safety intravenous set in which a lower structure of a transparent barrel is improved and thus a spherical ball in the transparent barrel is always and precisely floated up so that Ringer's solution is facilely administered when Ringer's solution is introduced into the transparent barrel and also, when the administration of the Ringer's solution is completed, the spherical ball blocks an outlet port so as to prevent a back flow of blood.

To achieve an aforementioned object of the present invention, there is provided a safety intravenous set in which a spherical ball for controlling a flow of Ringer's solution is provided in a transparent barrier for confirming a flow of the Ringer's solution, characterized in that, the transparent barrier of the intravenous set has a double-curved structure at a lower portion thereof, and also has a hemispherical settling portion for easily floating up the spherical ball when the Ringer's solution is introduced into the transparent barrier.

### BRIEF DESCRIPTION OF THE DRAWINGS

The above objects and other advantages of the present invention will become more apparent by describing in detail preferred embodiments thereof with reference to the attached drawings in which:

FIG. 1 is a cross-sectional view showing a status that Ringer's solution is injected from a safety intravenous set according to the present invention;

FIG. 2 is a cross-sectional view showing a status that the Ringer's solution is completely administered from the safety intravenous set according to the present invention;

FIG. 3 is a partially enlarged cross-sectional view of a transparent barrel when the Ringer's solution is introduced into the transparent barrel in an early stage according to the present invention;

FIG. 4 is an enlarged cross-sectional view of an A portion of FIG. 2;

FIG. 5 is a cross-sectional view of a spherical ball of a safety intravenous set according to the present invention; and

FIG. 6 is a schematic view of a conventional intravenous set.

### DETAILED DESCRIPTION OF THE PREFERRED EMBODIMENTS

Now, preferred embodiments of the present invention will be described in detail with reference to the annexed drawings. In the drawings, like elements having the same function are designated by identical reference numerals, and their repeated descriptions are intentionally omitted.

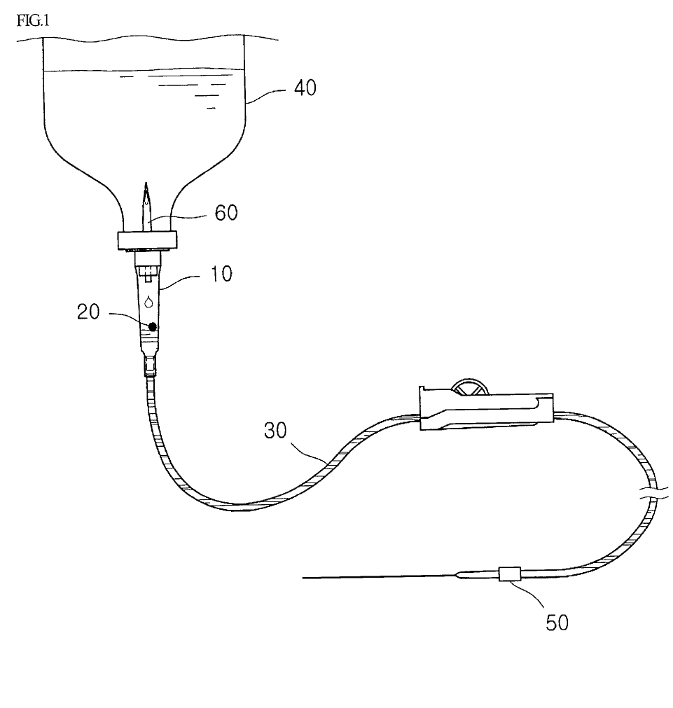

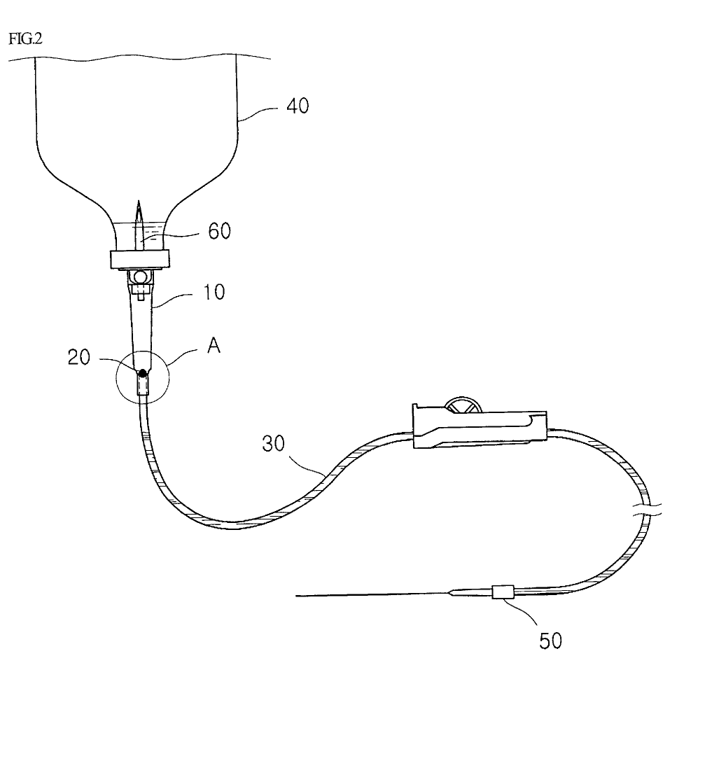

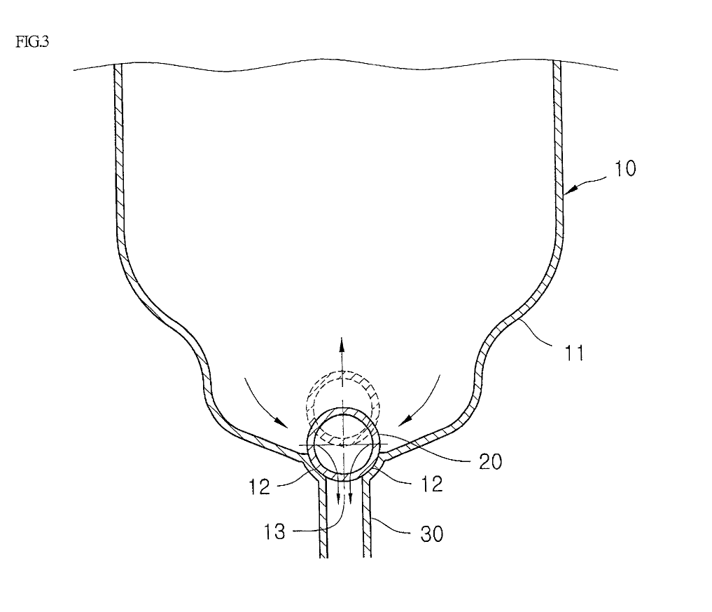

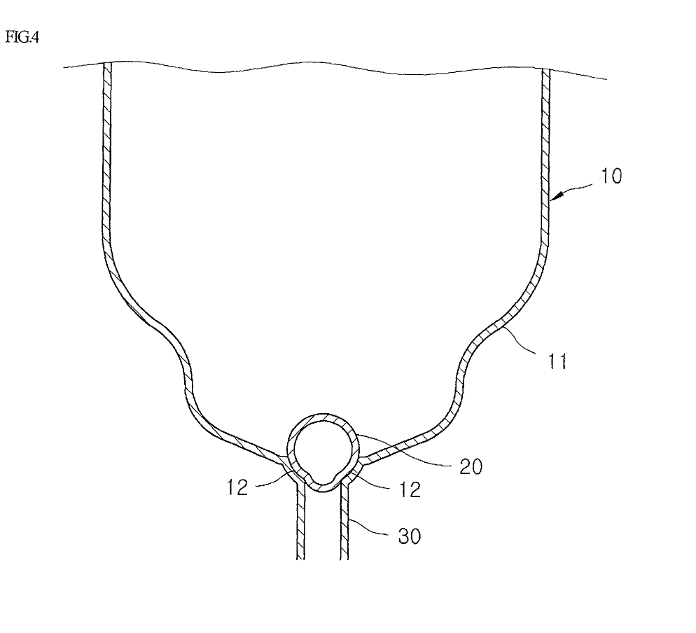

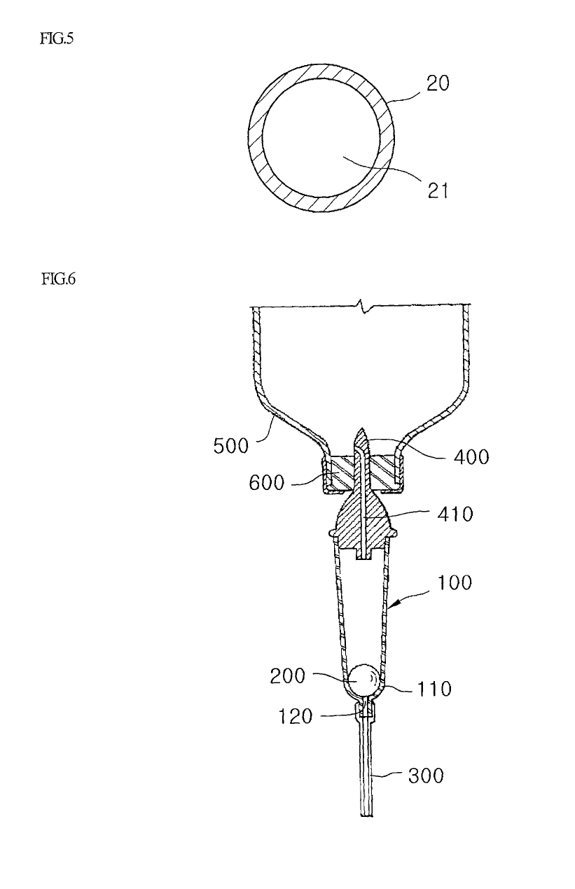

FIG. 1 is a cross-sectional view showing a status that Ringer's solution is injected from a safety intravenous set according to the present invention, FIG. 2 is a cross-sectional view showing a status that the Ringer's solution is completely administered from the safety intravenous set according to the present invention, FIG. 3 is a partially enlarged cross-sectional view of a transparent barrel when the Ringer's solution is introduced into the transparent barrel in an early stage according to the present invention, FIG. 4 is an enlarged cross-sectional view of an A portion of FIG. 2, and FIG. 5 is a cross-sectional view of a spherical ball of a safety intravenous set according to the present invention.

As shown in drawings, an intravenous set of the present invention, which is inserted into a Ringer bottle 40, is provided with a transparent barrel 10 having a needle rod 60 at an upper portion thereof, and a spherical ball 20 provided in the transparent barrel 10.

At a lower portion of the transparent barrel 10, there is formed a double-curved portion 11. And, at a portion come across the double-curved portions, there is formed a hemispherical ball settling portion 12 in which about a half or less of the spherical ball 20 is settled.

An outlet port 13 having a desired diameter is formed at a center portion of the hemispherical ball settling portion 12 to be connected with a supplying tube 30 for guiding Ringer's solution to a needle 50.

It is preferable that the spherical ball 20 is formed of harmless natural rubber and formed with a desired cavity 21 therein.

If the needle rod 60 is inserted into the transparent barrel 10, the Ringer's solution contained in the Ringer bottle 40 is introduced through the needle rod 60 into the transparent barrel 10.

Meanwhile, the spherical ball 20 is settled at the hemispherical ball settling portion 12 formed at the lower portion of the transparent barrel 10. If the Ringer's solution is introduced, the Ringer's solution is gathered in the double-curved portion 11. At this time, if a desired amount of the Ringer's solution is gathered, a liquid surface of the Ringer's solution is formed to be lower than a center of the spherical ball 20. Therefore, the spherical ball 20 always receives buoyancy.

Further, since a contact surface area between the spherical ball 20 and the hemispherical ball settling portion 12 is small, the spherical ball 20 can be easily floated up by small buoyancy.

As described above, if the spherical ball 20 is floated on the liquid surface of the Ringer's solution, the Ringer's solution is flowed through the outlet port 13 formed at the lower portion of the transparent barrel 10 and supplied through the supplying tube 30 connected with the needle 50 to the body.

Meanwhile, if the Ringer's solution is administered in the body, the amount of the Ringer's solution in the transparent barrel 10 is reduced. Thus, the spherical ball 20 is gradually settled down together with the Ringer's solution.

If the Ringer's solution is completely administered in the body, the spherical ball 20 is settled in the hemispherical ball settling portion 12. At this time, the Ringer's solution in the supplying tube 30 is flowed at a desired distance and then stopped by a closed pressure.

Since the cavity 21 filled with is formed in the spherical ball 20, when the Ringer's solution is flowed along the supplying tube 30, a part of the spherical ball 20 is inserted into the outlet port 13, thereby completely sealing the outlet port 13.

Therefore, when the Ringer's solution is completely administered, it is previously prevented that air is entered into the blood vessel or blood is back flowed through the supplying tube 30.

According to the safety intravenous set of the present invention, as described above, since the spherical ball in the transparent barrel is facilely floated up due to its own buoyancy when the Ringer's solution is introduced into the transparent barrel, a flowing operation of the Ringer's solution is precisely controlled. Also, when the Ringer's solution is completely administered in the body, since the outlet port is securely sealed by the spherical ball, the back flow of the blood is prevented, thereby increasing reliability of a product.

While the present invention has been described in detail, it should be understood that various changes, substitutions and alterations could be made hereto without departing from the spirit and scope of the invention as defined by the appended claims.

What is claimed is:

1. A safety intravenous set in which a spherical ball for controlling a flow of Ringer's solution is provided in a transparent barrier for confirming a flow of the Ringer's solution, characterized in that,
the transparent barrier 10 of the intravenous set has a double-curved structure 11 at a lower portion thereof, and also has a hemispherical settling portion for easily floating up the spherical ball when the Ringer's solution is introduced into the transparent barrier 10.
